(19) United States
(12) Patent Application Publication
(10) Pub. No.: US 2025/0339594 A1
(43) Pub. Date: Nov. 6, 2025

(54) SYSTEM AND METHOD FOR PROVIDING WATER FOR USE IN DIALYSIS

(71) Applicants: BAXTER INTERNATIONAL INC., Deerfield, IL (US); BAXTER HEALTHCARE SA, Glattpark (Opfikon) (CH); GAMBRO LUNDIA AB, Lund (SE)

(72) Inventors: Christian Vartia, Veberöd (SE); Vishnu Shyam, Lund (SE); Thomas Hertz, Lund (SE)

(21) Appl. No.: 18/868,548

(22) PCT Filed: May 23, 2023
(86) PCT No.: PCT/EP2023/063727

§ 371 Date: Nov. 22, 2024

(30) Foreign Application Priority Data
May 25, 2022 (SE) 2250620-8

(51) Int. Cl. A61M 1/16; A61M 1/34; C02F 1/44

(52) U.S. Cl. CPC A61M 1/1656; A61M 1/1696; A61M 1/3462; C02F 1/447

(57) ABSTRACT

A water supply system is operable to extract liquid water, EW, from waste fluid, WF, generated by a dialysis system and to provide the liquid water for use by the dialysis system. The water supply system comprises a dehumidifier sub-system and a humidifier sub-system. A gas stream is circulated between the sub-systems and is alternately humidified and dehumidified. The liquid water is extracted from the gas stream by the dehumidifier sub-system. The humidifier sub-system comprises a membrane distillation unit with a feed side and a draw side separated by a hydrophobic membrane. The waste fluid is supplied on the feed side and the gas stream is supplied on the draw side, and water vapor is transported from the waste fluid through the membrane via a difference in partial water vapor pressure between the feed and draw sides.

### TECHNICAL FIELD

The present disclosure relates generally to dialysis therapy, and in particular to a technique of providing water for use by a dialysis system when performing dialysis therapy.

### BACKGROUND ART

Dialysis therapy is undertaken to replace or supplement the normal blood-filtering function of the kidneys. It is used when the kidneys are not working well, which is known as kidney failure and includes acute kidney injury (AKI) and chronic kidney disease (CKD). Dialysis therapy involves removal of water from the body of the patient suffering from kidney failure, as well as exchange of solutes with the body. One example of dialysis therapy is extracorporeal (EC) blood therapy, in which blood is circulated outside of the patient and interfaced with one or more medical fluids. Modalities of extracorporeal blood therapy include hemodialysis (HD), hemofiltration (HF) and hemodiafiltration (HDF). Another example of dialysis therapy is peritoneal dialysis (PD), in which a medical fluid is infused into the peritoneal cavity of the patient to interface with the blood of the patient through the peritoneal membrane.

Medical fluids used in HD and PD are commonly known as dialysis fluids. In HF, the medical fluid is known as replacement fluid, since it is infused into the blood of the patient to replace fluid removed during therapy. In HDF, both dialysis fluid and replacement fluid are used.

Extracorporeal blood therapy by HD, HF or HDF is performed differently for treatment of patients with AKI compared to patients with CKD, by use of a different type of dialysis machine. Generally, compared to CKD patients, AKI patients are treated continuously over a longer period of time and at lower fluid flow rates. Such continuous treatment is commonly known as CRRT (Continuous Renal Replacement Therapy).

PD may be performed manually or be automated. In automated peritoneal dialysis (APD), the dialysis treatment is controlled by a machine, commonly known as a “cycler”. The machine is connected in fluid communication with the peritoneal cavity and is operated to control the flow of fresh dialysis fluid into the peritoneal cavity and the flow of spent dialysis fluid from the peritoneal cavity.

Over time, dialysis therapy consumes large quantities of medical fluid. In some modalities of dialysis therapy, pre-made medical fluid is delivered in prefilled bags to the point of care. For example, conventional PD is performed by use of prefilled bags. AKI machines are configured to use prefilled bags of medical fluid, by staff installing a prefilled bag before treatment and replacing the prefilled bag as required. On the other hand, CKD machines have integrated capability to generate medical fluid on-demand by mixing one or more concentrates with water, so-called on-line fluid generation. Recently, PD machines with integrated capability of on-line fluid generation have been proposed.

Local production of medical fluid at the point-of-care is attractive since it reduces the cost and environmental impact of transporting large amounts of ready-made medical fluid and the burden of storing and handling pre-filled bags. However, production of medical fluid requires access to purified water. Typically, a water purifier is connected to a tap water source, and a fluid generation unit is operated to mix one or more concentrates with the purified water to generate the medical fluid. Large amounts of tap water may be consumed. In PD, about 15 liters of medical fluid is consumed during each therapy session. In EC blood therapy, more than 100 liters of medical fluid may be consumed during a single therapy session. Correspondingly, large amounts of waste fluid is produced in dialysis therapy. The waste fluid may be directed to a drain at the point-of-care.

There is a general need to reduce the consumption of tap water during dialysis therapy.

There is also a general need to facilitate installation of a dialysis system that is configured to produce medical fluid. At present, the need for tap water and the need to dispose of the waste fluid restrict the installation. The tap water source and the drain may be located far away from the desired location of the dialysis system, requiring significant plumbing work and the use of extended tubing, which in turn increases the risk for leaks and consequential water damage.

### SUMMARY

It is an objective to at least partly overcome one or more limitations of the prior art.

One objective is to provide a technique for reducing the consumption of tap water by dialysis systems.

Another objective is to facilitate disposal of waste fluid that is generated by a dialysis system.

One or more of these objectives, as well as further objectives that may appear from the description below, are at least partly achieved by a water supply system, an arrangement comprising the water supply system, a computer-implemented method and a computer-readable medium according to the independent claims, embodiments thereof being defined by the dependent claims.

A first aspect is a water supply system for a dialysis system. The water supply system comprises: a first sub-system, which is configured to convert a first gas stream into a second gas stream by extracting liquid water from the first gas stream, and provide the liquid water for use by the dialysis system; a second sub-system, which is configured to process the second gas stream, by use of waste fluid from the dialysis system, to generate the first gas stream with increased humidity compared to the second gas stream; and a control arrangement, which is configured to jointly operate the first and second sub-systems generate a target amount of said liquid water. The second sub-system comprises a membrane distillation, MD, unit that defines a feed side and a draw side separated by a hydrophobic membrane. The MD unit is arranged to receive the waste fluid at an inlet on the feed side, and receive the second gas stream at an inlet on the draw side. The MD unit is configured to generate the first gas stream by transporting water vapor from the waste fluid into the second gas stream through the hydrophobic membrane via a difference in partial water vapor pressure between the feed side and the draw side.

The water supply system of the first aspect combines gas humidification with gas dehumidification to produce liquid water for use by a dialysis system. The first aspect is based on the insight that, by performing the gas humidification in the water supply system by membrane distillation, it is possible to make use of waste fluid produced by the dialysis system. Such a water supply system is operable to recycle at least part of the water in the waste fluid and will reduce the need for supplying tap water to operate the dialysis system. Further, since the membrane distillation removes water vapor from the waste fluid, the thus-processed waste fluid is decreased in volume, which facilitates its disposal. It is realized that the water supply system of the first aspect may significantly facilitate the handling of a dialysis system and may also facilitate its installation by reducing or eliminating the need for plumbing between a tap water source and/or a drain and the dialysis system and/or the water supply system.

In some embodiments, the control arrangement is configured to selectively operate the first sub-system to obtain at least part of the first gas stream from surrounding air, and supply at least part of the second gas stream to the surrounding air.

In some embodiments, the control arrangement is configured to selectively switch the system between a first mode, in which the system is configured to transfer the first and second gas streams between the first and second sub-systems in a closed loop, and a second mode, in which the system is configured to block said transfer and operate the first sub-system to obtain the first gas stream from the surrounding air and provide the second gas stream to the surrounding air.

In some embodiments, the control arrangement is configured to switch between the first and second modes based on at least one of a current water content of the surrounding air, availability of the waste fluid, availability of said liquid water, or a time schedule.

In some embodiments, the second sub-system further comprises a heating arrangement which is arranged upstream of the inlet on the feed side of the MD unit and is operable to heat the waste fluid.

In some embodiments, the heating arrangement comprises a heat transfer device, which is arranged to transfer thermal energy from the first gas stream, as generated by the MD unit, to the waste fluid.

In some embodiments, the second sub-system further comprises a WF sensor, which is arranged downstream of an outlet on the feed side of the MD unit to provide a measurement signal indicative of a concentration-related property of the waste fluid, and the control arrangement is configured to operate the second sub-system based on the measurement signal.

In some embodiments, the concentration-related property comprises a concentration, a density, a conductivity, a color, a transparency, or a refractive index.

In some embodiments, the second sub-system defines a recirculation path that includes the feed side of the MD unit, the second sub-system comprises a pumping device in the recirculation path, and the control arrangement is configured to operate the pumping device, based on the measurement signal, to recirculate the waste fluid through the feed side of the MD unit.

In some embodiments, the control arrangement is further configured to, based on the measurement signal, selectively operate a first flow controller to admit a first amount of waste fluid into the recirculation path and a second flow controller to expel a second amount of processed waste fluid from the recirculation path, wherein the processed waste fluid contains waste fluid that has been recirculated at least once through the feed side of the MD unit.

In some embodiments, the control arrangement is configured to, in sequence, operate the first flow controller to admit the first amount into the recirculation path, operate the pumping device to circulate at least the first amount through the feed side of the MD unit, and operate the second flow controller to expel the second amount from the recirculation path.

In some embodiments, the control arrangement is configured to, concurrently, operate the first flow controller to admit the first amount into the recirculation path and the second flow controller to expel the second amount from the recirculation path so that a difference between the first and second amounts substantially equals a third amount of water transported into the second gas stream through the hydrophobic membrane.

In some embodiments, the control arrangement is configured to selectively operate a supply arrangement to supply tap water to the recirculation path and operate the pumping device to circulate the tap water through the feed side of the MD unit.

In some embodiments, the water supply system further comprises an EW container, which is arranged to receive the liquid water from the first sub-system, and the control arrangement is configured to operate the first and second sub-systems in dependence of a fill level of the EW container, as indicated by a level sensor associated with the EW container.

In some embodiments, the water supply system further comprises a WF container, which is arranged for intermediate storage of the waste fluid and is fluidly connected to the inlet on the feed side of the MD unit.

In some embodiments, the WF container is associated with a sterilization device which is operable to sterilize the WF container.

In some embodiments, the first and second gas streams comprise air.

In some embodiments, the control arrangement is configured to determine first settings of the first sub-system to achieve the target amount, and determine second settings of the second sub-system based on the first settings, wherein the second settings define a water content and a flow rate of the first gas stream as generated by the second sub-system.

In some embodiments, the first sub-system comprises a desiccant, which is arranged to adsorb and/or absorb moisture from the first gas stream and which is processed by the first sub-system to extract the liquid water from the desiccant.

In some embodiments, the desiccant is configured to have a high selectivity towards water.

In some embodiments, the liquid water that is extracted from the first gas stream has a conductivity of less than 10 μS/cm, and preferably less than 5 μS/cm or 1 μS/cm.

In some embodiments, the first sub-system comprises a cooling element, which is configured to cool the first gas stream to extract the liquid water from the first gas stream by condensation.

A second aspect is an arrangement comprising the water supply arrangement of the first aspect, or any embodiment thereof, and a dialysis system which is configured to receive medical fluid for use in dialysis therapy performed by the dialysis system and to produce waste fluid that is at least partly generated from the medical fluid during the dialysis therapy, wherein the dialysis system is fluidly connected to transfer the waste fluid to the water supply system.

In some embodiments, the dialysis system comprises a fluid preparation sub-system which is configured to receive at least part of the liquid water that is provided by the water supply system and generate the medical fluid by mixing said at least part of the liquid water with one or more concentrates.

In some embodiments, the dialysis system is configured to generate a portion of the waste fluid during a cleaning operation of the dialysis system, the dialysis system being configured to perform the cleaning operation by use of a portion of the liquid water that is provided by the water supply system.

A third aspect is a computer-implemented method of providing water for use by a dialysis system. The method comprises: operating a first sub-system to convert a first gas stream into a second gas stream by extracting liquid water from the first gas stream; providing the liquid water for use by the dialysis system; and operating a second sub-system, in coordination with the first sub-system, to process the second gas stream, by use of waste fluid from the dialysis system, to generate the first gas stream with increased humidity compared to the second gas stream. The operating the second sub-system comprises: supplying the waste fluid at an inlet on a feed side of a membrane distillation, MD, unit, and supplying the second gas stream at an inlet on a draw side of the MD unit, the draw side being separated from the feed side by a hydrophobic membrane, the MD unit being configured to generate the first gas stream by transporting water vapor from the waste fluid into the second gas stream through the hydrophobic membrane via a difference in partial water vapor pressure between the feed side and the draw side.

The embodiments of the first aspect may be adapted as embodiments of the third aspect.

A fourth aspect is a computer-readable medium comprising program instructions, which when executed by processing circuitry causes the processing circuitry to perform the method of the second aspect, or any embodiment thereof. The computer-readable medium may be a non-transitory medium or a propagating signal.

Still other objectives, aspects, embodiments, and technical effects, as well as features and advantages may appear from the following detailed description, from the attached claims as well as from the drawings.

### BRIEF DESCRIPTION OF THE DRAWINGS

FIGS. 1A-1C are schematic views of an example water supply system combined with various dialysis systems.

FIG. 2 is a block diagram of an example water supply system.

FIGS. 3A-3B are flow charts of example operating methods for the water supply system in FIG. 2.

FIG. 4 is a block diagram of an example dehumidifier unit for extraction of liquid water from a gas stream.

FIGS. 5A-5B are block diagrams of water supply systems configured for batch processing and continuous processing, respectively, of waste fluid.

### LIST OF ABBREVIATIONS

DH Dehumidification
DHU Dehumidifier
DIA Incoming air stream
DOA Outgoing air stream
EIA Incoming environment air
EOA Outgoing environment air
EC Extracorporeal
EW Extracted liquid water
FWF Final waste fluid
HD Hemodialysis
HDH Humidification-dehumidification
Hdi Inlet humidity
HDF Hemodiafiltration
Hdo Outlet humidity
HF Hemofiltration
HU Humidifier
MD Membrane distillation
MF Medical fluid
MOF Metal-organic framework
PC Peritoneal cavity
PD Peritoneal dialysis
RH Relative Humidity
TW Tap water
WF Waste fluid
WSS Water supply system

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements, whereas the term a “set” of elements is intended to imply a provision of one or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

As used herein, “dialysis therapy” refers to any therapy that replaces or supplements the renal function of a patient by use of a medical fluid. Dialysis therapy includes, without limitation, extracorporeal blood therapy and peritoneal dialysis therapy.

As used herein, “medical fluid” refers to any fluid that is consumed as a result of dialysis therapy. Medical fluid includes, without limitation, dialysis fluid for infusion into the peritoneal cavity during peritoneal dialysis therapy, dialysis fluid for supply to a dialyzer during EC blood therapy, replacement fluid and substitution fluid for infusion into blood during EC blood therapy, priming fluid, and fluid for disinfection and/or cleaning of the dialysis system.

As used herein, “sterilization” refers to any process that substantially removes, kills, or deactivates microorganisms and other biological agents. In the context of the present disclosure, no distinction is made between sterilization, disinfection, and sanitization. Sterilization may involve applying one or more of heat, chemicals, irradiation, high pressure, or filtration.

As used herein, “purification” refers to a process of substantially removing undesirable chemicals, biological contaminants, suspended solids, and gases from water, for the purpose of providing water with an acceptable purity for use in medical fluid.

As used herein, “priming” refers to a process of removing air and/or possible fragments of remaining sterilizing agents or other residuals, from fluid paths within a dialysis system before a treatment session is started. Priming involves flushing the fluid paths with a human-compatible liquid.

As used herein, “tap water” refers to water that is provided through a water dispenser valve (“tap”) connected to indoor plumbing. Tap water is also known as faucet water, running water, or municipal water and is commonly used for drinking, cooking, washing, and toilet flushing.

Like reference signs refer to like elements throughout.

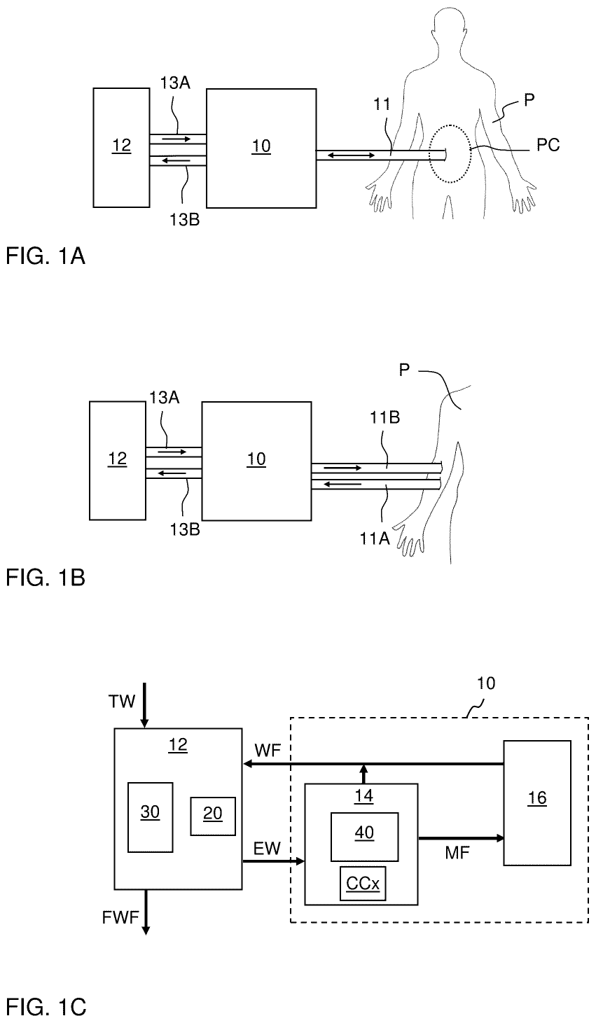

The present disclosure relates to a technique of providing water for use in a dialysis system. The technique is applicable to any type of dialysis system, including systems for peritoneal dialysis (PD) therapy or extracorporeal (EC) blood therapy. For context only, water usage in relation to PD therapy and EC blood therapy will be briefly exemplified and discussed with reference to FIGS. 1A-1B.

FIG. 1A is a generic overview of a system for PD therapy. The system comprises a dialysis system 10, which is fluidly connected to the peritoneal cavity (PC) of a patient P. As indicated by a double-ended arrow, the dialysis system 10 is operable to convey fresh dialysis fluid into the PC and to receive spent dialysis fluid from the PC on fluid path 11. The fluid path 11 may be defined by tubing that connects to an implanted catheter (not shown) in fluid communication with the PC. The dialysis system 10 may be configured for any type of PD therapy. In one example, the dialysis system 10 comprises a dialysis machine (“cycler”) that performs the dialysis therapy. The dialysis system 10 is fluidly connected to receive water from a water supply system (WSS) 12 on a first fluid path 13A and to supply waste fluid to the WSS 12 on a second fluid path 13B. In embodiments described hereinbelow, the waste fluid is used by the WSS 12 to generate water. The water as supplied by the WSS 12 may be used by the dialysis system 10 to produce the dialysis fluid by mixing the water with one or more concentrates. The water may also be used in maintenance operations such as cleaning (rinsing), sterilization or priming of the dialysis system 10. For example, the dialysis system 10 may be configured to produce a dedicated maintenance fluid for the respective maintenance operation by use of the water from the WSS 12. Alternatively, such a maintenance fluid may be supplied from separate source, for example a pre-filled bag.

FIG. 1B is a generic overview of a system for EC blood therapy. The WSS 12 may be the same as in FIG. TA. The dialysis system 10 is fluidly connected to the vascular system of a patient P on a fluid path. In the illustrated example, the fluid path comprises tubing 11A for blood extraction and tubing 11B for blood return. As indicated by arrows, the dialysis system 10 is operable to draw blood from the patient P through tubing 11A, process the blood, and return the processed blood to the patient through tubing 11B. The respective tubing 11A, 11B is connected to an access device (for example a catheter, graph or fistula, not shown) in fluid communication with the vascular system of the patient P. The dialysis system 10 may be configured to process the blood by any modality of EC blood therapy, such as HD, HF or HDF. Depending on modality, dialysis fluid and/or replacement fluid is consumed during such therapy. The dialysis system 10 may be configured to generate dialysis fluid and/or replacement fluid, as needed, by mixing purified water with one or more concentrates. The dialysis system 10 is fluidly connected to receive water from the WSS 12 on a first fluid path 13A and to supply waste fluid to the WSS 12 on a second fluid path 13B. By analogy with FIG. TA, the dialysis system 10 may also produce maintenance fluid by use of the water from the WSS 12.

FIG. 1C is a more detailed view of a system comprising a dialysis system 10 and a WSS 12. The dialysis system 10 comprises a preparation sub-system 14 and a therapy sub-system 16. The therapy sub-system 16 is configured to receive medical fluid (MF) and perform dialysis therapy by use of the MF. It is understood that the therapy sub-system 16 is configured to perform a specific type of dialysis therapy, such as PD therapy or EC blood therapy, of any modality. It is also realized that MF may be a dialysis fluid or a replacement fluid, or a combination thereof, depending on dialysis therapy. During dialysis therapy, the therapy sub-system 16 produces waste fluid (WF), which comprises waste, toxins and excess water from the patient. If dialysis fluid is used in the dialysis therapy, the waste fluid comprises used dialysis fluid, also known as “spent dialysis fluid”, which includes at least part of the waste, toxins and excess water. The waste fluid may also comprise MF that has been discarded without being used, as well as maintenance fluid that has been used in a maintenance operation (“used maintenance fluid”).

The preparation sub-system 14 comprises a mixing arrangement 40, which is configured to mix water with one or more concentrates (CCx) to produce the medical fluid, MF. MF may be produced in batches or on-demand by the preparation sub-system 14. Here, “on-demand” implies that the MF production rate matches the MF consumption rate in the therapy sub-system 16. The respective concentrate may be in the form of a liquid or a powder. Mixing arrangements for MF production are well-known in the art and need not be described in detail herein. It is to be understood that preparation sub-system 14 may comprise any combination of conventional components for temperature adjustment, degassing, etc. As indicated by an arrow in FIG. 1C, the fluid preparation sub-system 14 may also output WF, which may include used maintenance fluid and discarded MF.

As indicated in FIG. 1C, the WSS 12 is configured to receive WF from the dialysis system 10 and process the WF for extraction of liquid water, denoted EW. The WSS 12 is arranged to provide EW to the preparation sub-system 14 for use in producing MF and, optionally, maintenance fluid. The WF processing results in final waste fluid (FWF), which is output from the WSS 12 for disposal. As indicated, the WSS 12 may also receive tap water (TW) for use in the EW production. The WSS 12 comprises a combination of a dehumidifier (DHU) sub-system 20 and a humidifier (HU) sub-system 30, which are jointly operated to extract EW from WF. As described in more detail below, the HU sub-system 30 utilizes membrane distillation (MD) to humidify a gas by use of the waste fluid. The thus-humidified gas is conveyed to the DHU sub-system 20, which is configured to produce EW from the humidified gas. In other words, the gas is utilized as a medium for transporting water vapor from the HU sub-system 30 to the DHU sub-system 20. Typically, a large proportion of WF is water, and this water is available for extraction by MD. The WSS 12 is thereby capable of producing water for the dialysis system 10 with minimum, or even no, use of tap water.

Membrane distillation is a separation process which is driven by phase change. A hydrophobic membrane (MD membrane) presents a barrier for the liquid phase, allowing the vapor phase to pass through the MD membrane's pores. The driving force of the separation process is a difference in partial vapor pressure between opposite sides of the MD membrane, commonly known as a feed side and a draw side (or permeate side), respectively. In HU sub-system 30, waste fluid is provided on the feed side, and a gas is provided on the draw side. By controlling the difference in partial water vapor pressure between the feed and draw sides, water vapor is transported from the waste fluid through the MD membrane into the gas. By proper design of the MD membrane, effectively all non-volatile substances, such as salts, may be retained on the feed side. The gas is driven to flow past the MD membrane on the draw side, thereby increasing the amount of water that may be transported through the MD membrane per unit time. This type of MD technique is known as Sweeping Gas MD (SWGMD) in the art.

The water vapor separation in the HU sub-system 30 will reduce the residual volume of waste fluid to be handled as a result of a therapy session, denoted FWF in FIG. 1C. For example, the volume ratio of FWF to WF may be in the range of 0.1-0.5. The reduced need of tap water and the small volume of FWF have the potential of facilitating installation. For example, the need for a permanent fluid connection between the WSS 12 and a water tap may be obviated. Any required tap water may be manually supplied to the WSS 12 by the user without difficulty. Likewise, the need for a permanent fluid connection to a drain may also be obviated. For example, the FWF may be collected in a container, which is manually drained by the user.

The mobility of the dialysis systems may also be increased by use of the WSS as described herein, by reducing the amount of fluid that needs to be transported together with the dialysis system to render it operable. In addition to concentrate(s) used by the dialysis system, only a fraction of the total amount of the water that is consumed during a therapy session may be transported, if the WSS is operable to extract water from the waste fluid as it is produced by the dialysis system.

Depending on implementation, the water that is extracted by the DHU sub-system 20 may have a sufficient purity to comply with quality requirements for water to be included in medical fluid, for example according to ISO 23500-3. If not, the WSS 12 and/or the dialysis system 10 may comprise a dedicated purification device for processing the water from DHU sub-system 20 before it is used by the mixing arrangement 40. The dedicated purification device may apply any conventional water purification technique to meet the above-mentioned quality requirements. It is also to be understood that the WSS 12 and/or the dialysis system 10 may comprise sterilization equipment to ensure that the medical fluid complies with microbial requirements, for example according to aforesaid ISO 23500-3.

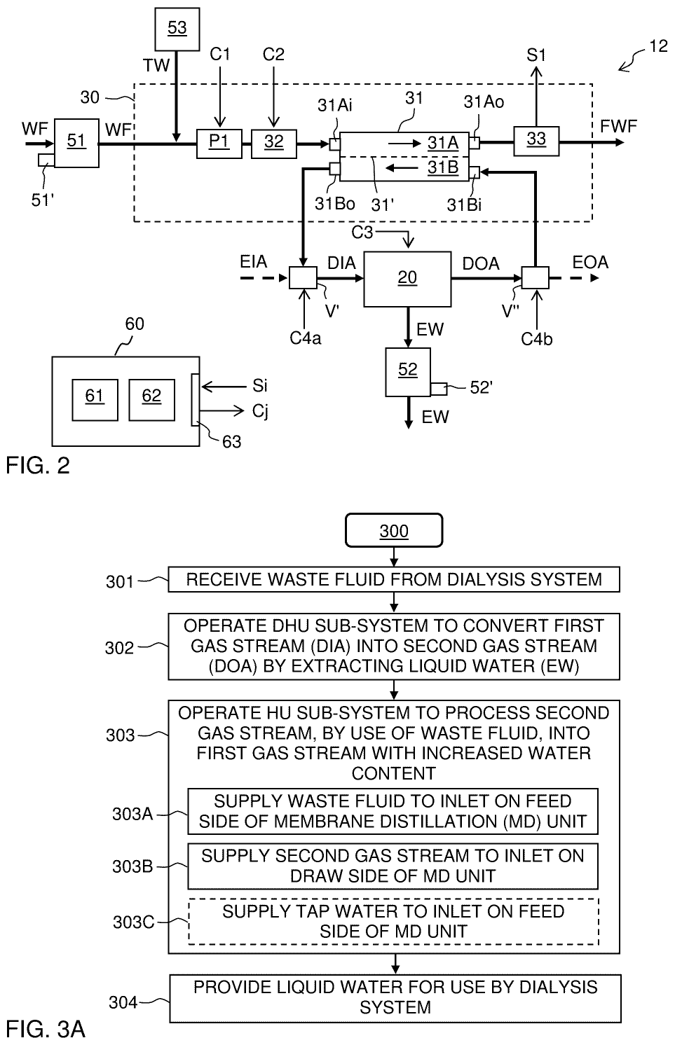

FIG. 2 is a block diagram of an example WSS 12 comprising a HU sub-system 30 and a DHU sub-system 20. In the illustrated example, the HU sub-system 30 is fluidly connected to the DHU sub-system 20 to form a closed gas loop, in which humified gas (DIA) generated by the HU sub-system 30 is supplied to the DHU sub-system 20 for dehumidification, resulting in extracted water (EW) and dehumidified gas (DOA), which is supplied to the sub-system 30 for humification, again resulting in the humidified gas (DIA). The gas is driven to circulate in the closed loop by a pumping device (not shown).

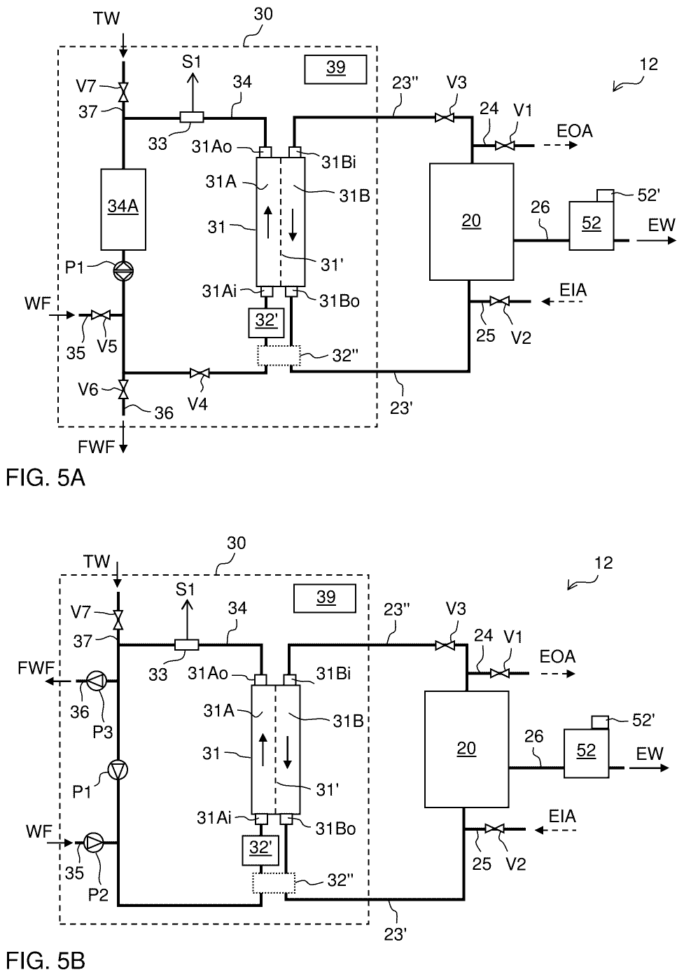

A control device 60 (“main controller”) is arranged to jointly operate the sub-systems 20, 30 to produce a target amount of EW. The target amount may be defined as a specified flow rate of EW, a specified amount of EW over a specified time period, or a specified or non-specified amount of EW produced when the sub-systems 20, 30 are operated at maximum capacity. The main controller 60 is configured to receive measurement signals, which are indicative of the operation of the WSS 12 and are represented as Si, and provide control signals, which control the operation of the WSS 12 and are represented as Cj. The WSS 12 may or may not comprise sub-controllers, which are operated under control of the main controller 60. For example, the DHU sub-system 20 may comprise a local controller (209 in FIG. 4), which is configured to control the operation of the DHU sub-system 20 subject to a control signal C3 from the main controller 60. Similarly, the HU sub-system 30 may comprise a corresponding local controller (39 in FIGS. 5A-5B). The main controller 60 may be configured to generate the control signals Cj in accordance with a control program comprising computer instructions. The main controller 60 comprises circuitry that includes one or more processors 61 and computer memory 62. The control program may be stored in the memory 62 and executed by the processor(s) 61. The control program may be supplied to the main controller 60 on a computer-readable medium, which may be a tangible (non-transitory) product (e.g., magnetic medium, optical disk, read-only memory, flash memory, etc.) or a propagating signal. In the illustrated example, the main controller 60 comprises a signal interface 63 for input of signals Si and output of signals Cj.

The HU sub-system 30 comprises a membrane distillation (MD) unit 31. The MD unit 31 comprises a feed side 31A and a draw side 31B separated by an MD membrane 31′. The different sides may also be referred to as compartments. The membrane 31′ is a hydrophobic microporous membrane, which may be configured in accordance with established knowledge, for example as described in the article “Membrane distillation: A comprehensive review” by Alkhudiri et al. in Desalination, vol. 287, pp 2-18 (2012), which is incorporated herein by reference. The geometry of the membrane 31′ may be flat-sheet, tubular or hollow fiber. The MD unit 31 is arranged to carry a liquid fluid on the feed side 31A and a gaseous fluid on the draw side 31B. The fluid flows are typically counter-current within the MD unit 31, as shown by arrows, but may alternatively be co-current. The respective fluid flow through the MD unit 31 may be continuous or intermittent. In some embodiments, exemplified in FIG. 2, the waste fluid passes only once through the feed side 31A (“single-pass configuration”). In other embodiments, exemplified in FIGS. 6A-6B and described further below, the waste fluid is re-circulated through the feed side 31A at least once (“multi-pass configuration”). As used herein, “recirculated at least once” implies that the waste fluid passes the MD unit 31 at least twice.

The MD unit 31 comprises at least one input port 31Ai (one shown) and at least one output port 31Ao (one shown) on the feed side 31A, and at least one input port 31Bi (one shown) and at least one output port 31Bo (one shown) on the draw side 31B. The MD unit 31 is configured to receive the waste fluid, WF, through the input port(s) 31Ai on the feed side 31a and to receive a gas, DOA, through the input ports(s) 31Bi on the draw side 31B. As understood from the above, water vapor is transported from WF to the gas through the membrane 31′, resulting in humidified gas DIA, which leaves the draw side 31B through the outlet port(s) 31Bo. The incoming waste fluid will become more concentrated by the MD process. In the illustrated example with a single-pass configuration, the waste fluid that leaves the feed side 31A through the outlet port(s) 31Ao forms final waste fluid, FWF, which is provided for disposal.

The HU sub-system 30 further comprises a pump P1 for driving the flow of waste fluid, WF, through the feed side 31A. In the illustrated example, the pump P1 is arranged upstream of the inlet port(s) 31Ai but may alternatively or additionally be arranged downstream of the outlet port(s) 31Ao.

In the illustrated example, the HU sub-system 30 further comprises a heating arrangement 32 which is arranged upstream of the inlet port(s) 31Ai. The heating arrangement 32 is operable to heat the waste fluid before it enters the feed side 31A. The heating will increase the partial pressure of water vapor on the feed side 31A and thereby promote the transport of water vapor through the membrane 31′. The heating arrangement 32 may comprise an electrical heater. Although not shown in FIG. 2, the heating arrangement 32 may further include a temperature sensor downstream of the electrical heater to provide temperature feedback for controlled heating.

In the illustrated example, the HU sub-system 30 further comprises a “WF sensor” 33, which is arranged downstream of the outlet port(s) 31Ao. The WF sensor 33 is operable to measure a concentration-related property of the waste fluid that leaves the feed side 31A of the MD unit 31 and provide a corresponding measurement signal S1. The concentration-related property correlates with or represents the concentration of one or more substances in the waste fluid and is thus indicative of the water transport in the MD unit 31. The WF sensor 33 may be configured to provide relative or absolute measurements. In one embodiment, the WF sensor 33 is a concentration sensor, for example configured to measure sodium concentration, or a conductivity sensor. In another embodiment, the WF sensor 33 is configured to measure density. In one example, density is determined by measuring the weight of a predefined volume of the waste fluid that leaves the MD unit 31. In another embodiment, the WF sensor 33 is configured to measure a color, a transparency, or a refractive index of the waste fluid.

The operation of the HU sub-system 30 is controlled by the main controller 60, optionally via a local controller (cf. 39 in FIGS. 5A-5B) in the HU sub-system 30. As shown, the HU sub-system 30 is at least partly controlled by a control signal C1 for the pump P1 and a control signal C2 for the heating arrangement 32 (if present). The control signals C1, C2 may be generated at least partly based on the signal Si from the WF sensor 33. The operation of the DHU sub-system 20 is likewise controlled by the main controller 60, by one or more control signals. In FIG. 2, it is assumed that the DHU sub-system 20 comprises a local controller which is operated based on a control signal C3 from the main controller 60.

In FIG. 2, the WSS 12 comprises a first container 51 (“WF container”), which is fluidly connected to receive waste fluid from the dialysis system. The WF container 51 is arranged to hold an intermediate supply of waste fluid for use by the HU sub-system 30. To this end, the WF container 51 is fluidly connected to the inlet port(s) 31Ai. The provision of the WF container 51 decouples the operation of the HU sub-system 30 from the production of waste fluid by the dialysis system. For example, WF may be accumulated in the container 51 during a therapy session for later use during the same or another therapy session. The WF container 51 may be configured as a permanent part of the WSS 12 or as a disposable unit, which is discarded a predefined time after installation or after a predefined number of therapy sessions. In the illustrated example, a sterilization device 51′ is associated with the WF container 51. The sterilization device 51′ is selectively operable, by the main controller 60, to sterilize the internal compartment of the container 51 and/or the waste fluid therein. The sterilization device 51′ is configured to use any suitable sterilization technique, such as application of heat or radiation (for example, UV radiation). Alternatively or additionally, the walls of the internal compartment of the container 51 may be lined with an antimicrobial material, for example a plastic material.

Although not shown in FIG. 2, the WSS 12 may comprise two or more WF containers 51, for example one container for holding waste fluid of high concentration such as spent medical fluid or discarded medical fluid, and one container for holding waste fluid of low concentration such as rinse fluid or priming fluid. The WSS 12 may be selectively operable, for example by a suitable valve arrangement (not shown), to fluidly connect any one of the WF containers to the inlet port(s) 31Ai.

In FIG. 2, the WSS 12 comprises a second container 52 (“EW container”), which is arranged to receive EW from the DHU sub-system 20. The EW container 52 is arranged to hold an intermediate supply of EW for use by the dialysis system. The EW container 52 thereby decouples the operation of the DHU sub-system 20 from the consumption of water by the dialysis system. The EW container 52 may be a permanent part or a disposable unit. In the illustrated example, a level sensor 52′ is provided to indicate the amount of liquid water in the EW container 52. In some embodiments, the main controller 60 is configured to operate the sub-systems 20, 30 in dependence of the fill level of the reservoir 52. For example, the sub-systems 20, 30 may be controlled to ensure that the amount of water in the EW container 52 exceeds a minimum level. The minimum level may be fixed or vary over time, for example during the course of a therapy session.

In FIG. 2, the WSS 12 comprises a third container (“TW container”) 53, which is fluidly connected to the inlet ports(s) 31Ai to supply tap water into the feed side 31A of the MD unit 31, either separated from or together with WF. The WSS 12 may be operated to supply TW from the TW container 53 to supplement the WF, for example if the available amount of WF in the WF container 51 is deemed to be insufficient in view of an EW production target. Thus, tap water is only used occasionally and the TW container 53 may be manually filled with tap water by the user.

Although not shown in FIG. 2, the EW container 52 and/or TW container 53 may be provided with a respective sterilization device and/or be internally lined with an antimicrobial material, similar to the WF container 51. Further, a respective level sensor may be provided to indicate the amount of fluid within the WF container 51 and/or the TW container 53, similar to the level sensor 52′ of the EW container 52.

In FIG. 2, the WSS 12 further comprises a valve arrangement, represented by a first and second valves V′, V″ upstream and downstream, respectively, of the DHU sub-system 20. The valve arrangement is operable to switch the WSS 12 between a HDH mode (Humidification-DeHumidification) and a DH mode (DeHumidification).

In the HDH mode, as shown by solid arrows, the DHU sub-system 20 is fluidly connected to the HU sub-system 30 in a closed loop so that the same gas is alternately and repeatedly humidified in the HU sub-system 30 and dehumidified in the DHU sub-system 20. Thereby, water vapor is extracted from WF in the MD unit 31 and materialized as liquid water in the DHU sub-system 20.

In the DH mode, the DHU sub-system 20 is fluidly decoupled from the HU sub-system 30, which is instead operated to receive and dehumidify “environment air”, i.e. air from the surroundings, EIA. The dehumidified air, EOA, is output to the surroundings. Thus, in the DH mode, DIA is equal to EIA and DOA is equal to EOA. In the DH mode, the sub-system 30 may be disabled to save power, for example by deactivating the pump P1 and the heating arrangement 32.

In FIG. 2, the respective valve V′, V″ is a 3-way valve which is operable to selectively open different passageways in dependence of a control signal C4a, C4b, which is generated by the main controller 60 or the local controller 209 in FIG. 4. The valve arrangement in FIG. 2 is merely an example and many alternative configurations are available to the skilled person.

The DH mode is optional but may be used to supplement the water extraction from the waste fluid, for example when there is a shortage of WF or when the extracted water from WF is deemed to be insufficient to meet the needs of the dialysis system.

It is also conceivable to combine the HDH and DH modes, so that the WSS 12 is operated to both circulate gas between the sub-systems 20, 30 and to pass surrounding air through the DHU sub-system 20 for dehumidification. In such a combination, the circulating gas is air and part of this air will be refreshed by the air exchange represented by EIA and EOA in FIG. 2. In other words, EIA will form part of DIA, and EOA will form part of DOA. However, volatile substances in the WF may pass through the membrane 31′. If such volatile substances are emitted to the surroundings, an unpleasant or at least unfamiliar smell may be experienced by the user. At least for this reason, it is currently believed that exclusive switching between HDH and DH modes is a better option.

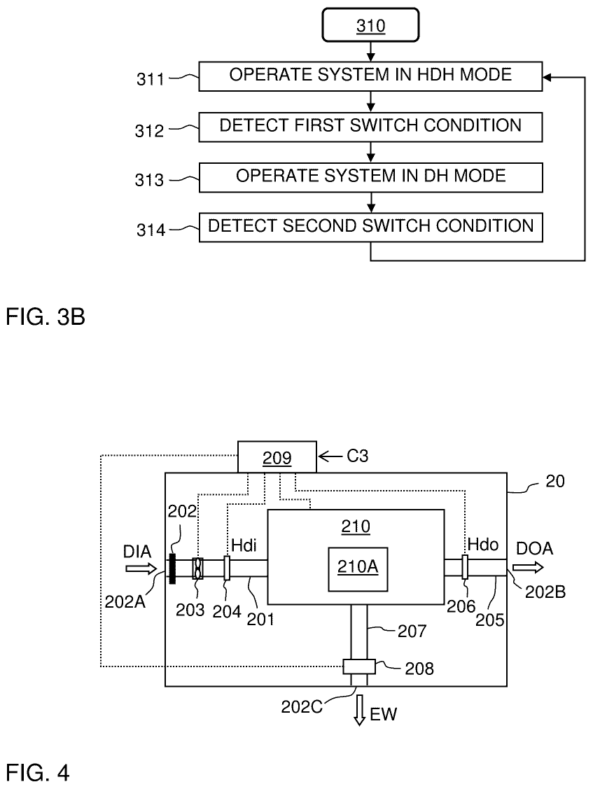

FIG. 3A is flow chart of a method 300 of operating a WSS 12 in the HDH mode. The method 300 comprises steps 301-304, which will be described with reference to the WSS 12 in FIG. 2.

In step 301, waste fluid is received from the dialysis system. Step 301 may be performed whenever WF is produced by the dialysis system. In the example of FIG. 2, the incoming WF is accumulated in the WF container 51.

Steps 302 and 303 are performed concurrently by circulating a gas in a closed loop between the sub-systems 20, 30 (solid arrows in FIG. 2). In step 302, the DHU sub-system 20 is operated to convert a first gas stream into a second gas stream by extracting liquid water (EW) from the first gas stream. In FIG. 2, step 302 involves dehumidification of the incoming humidified gas stream (DIA) to produce the outgoing dehumidified gas stream (DOA). In step 303, the HU sub-system 30 is operated to process the second gas stream, produced by the DHU sub-system 20, to increase its humidity by use of the waste fluid. The second gas stream thereby forms the first gas stream which is output for receipt by the DHU sub-system 20. In FIG. 2, step 303 involves humidification of incoming gas, to convert the dehumidified gas stream (DOA) into the humidified gas stream (DIA).

As understood from the example structure in FIG. 2, step 303 involves use of an MD unit 31. Consequently, as indicated in FIG. 3A, step 303 comprises a step 303A of supplying WF at an inlet 31Ai on the feed side 31A of the MD unit 31, and a step 303B of supplying the second gas stream (DOA) at an inlet 31Bi on the draw side 31B of the MD unit 31.

In step 304, EW is provided for use by the dialysis system. Step 304 may be performed whenever water is needed by the dialysis system. In the example of FIG. 2, EW is supplied from the EW container 52, in which the EW is accumulated during operation of the DHU sub-system 20.

In the example of FIG. 2, steps 302 and 303 are performed by the main controller 60, which controls the operation of the sub-systems 20, 30 by the control signals C1, C2, C3, C4a, C4b, which are generated at least partly based on the measurement signal S1. Steps 301 and 304 may be performed by the main controller 60 or a separate controller of the dialysis system.

The gas that is circulated between the sub-systems 20, 30 may be selected to optimize DHU-HU performance if the WSS 12 is configured to only operate in the HDH mode, in which the gas is confined to a closed fluid circuit. However, if the WSS 12 is operable to process air from the surroundings, for example in the DH mode, the WSS 12 will circulate air through the sub-systems 20, 30 in the HDH mode. For practical reasons, it may be advantageous to circulate air even if the WSS 12 is configured to only operate in the HDH mode.

FIG. 3B is a flow chart of a control procedure 310 that may be performed by the main controller 60 to selectively set the WSS 12 in the HDH mode or the DH mode. In step 311, the WSS 12 is operated in the HDH mode, which is the default mode. In step 312, a first switch condition is detected, which causes the WSS 12 to be switched to the DH mode. When the WSS 12 is then operated in the DH mode, according to step 313, a second switch condition is detected in step 314, causing the WSS 12 to be switched back to the HDH mode. As indicated by an arrow from step 314 to step 311, the WSS 12 may be switched back and forth between the HDH and DH modes any number of times during operation.

The switching between modes may be pre-scheduled. Thus, the first and second switch conditions may be given by time points of a time schedule. For example, the pre-scheduling may be based on user preferences or noise considerations. It should be understood that the DH mode is likely to produce more noise than the HDH mode as a result of the exchange of air with the surroundings. For example, it may be undesirable to operate the WSS 12 in DH mode at night if the WSS 12 is located in the premises of the user. The switching may also be performed dynamically based on sensed properties of the WSS 12 or its surroundings. For example, a switch from the HDH mode to the DH mode may be triggered (step 311) by an insufficient availability of WF. In FIG. 2, the availability of WF may be given by the amount of WF in the WF container 51. Conversely, a switch from the DH mode to the HDH mode may be triggered (step 314) when the availability of WF is again sufficient. In another example, the switching may be triggered based on the amount of water in the surrounding air, for example represented by a relative humidity (RH) measured by the WSS 12 (cf. sensors 204, 206 in FIG. 4, below). For example, a switch from the DH mode to the HDH mode may be triggered (step 314) when the humidity is below a limit value, so as to maintain an acceptable humidity in the premises of the WSS 12. Conversely, a switch from the HDH mode to the DH mode may be triggered (step 312) when the available amount of water in the surrounding air is above a further limit value. The amount of water may be given by RH, optionally in combination with room size, which may be estimated by the WSS 12 or entered by the user. In further example, the switch in step 312 and/or step 314 may be performed (pre-scheduled or dynamically) based on historical data representing the operation of the WSS 12, the propensity for membrane fouling in the MD unit 31, etc.

By the provision of the WF container 51, it is possible to store WF for later use, should not all WF be processed into EW during a dialysis session. For example, if a dialysis session is performed at nighttime, at least part of the WF may be saved and processed for water extraction after the treatment, during daytime. Thus, all of the WF that is produced during a dialysis session need not be processed during this dialysis session.

It may be beneficial to distribute the operation of the WSS 12 in the DH mode over the day to reduce the drying effect on the surrounding air and also to increase the amount of water that is extracted by the DHU sub-system 20. If the WSS 12 is operated in the HDH mode for a prolonged period of time, a significant amount of available water in the surrounding air will be unused and potentially be ventilated from the premises. By intermittently switching to the DH mode, it is possible to utilize the water in the surrounding air without significantly impacting the air humidity in the surroundings. By using DH mode for shorter time periods with HDH mode active in-between, the surrounding air humidity is allowed to recover.

Reverting to FIG. 3A, it is seen that step 303 may comprise an optional step 303C of selectively admitting tap water (TW) at an inlet 31Ai on the feed side 31A of the MD unit 31. TW may be admitted through the same inlet as WF, as shown in FIG. 2, or through a separate inlet. Step 303C may be performed based on the availability of WF and/or EW in relation to an actual or expected EW consumption by the dialysis system. By providing TW to the feed side 31A, TW is seamlessly introduced into the existing water extraction process performed by the sub-systems 20, 30 and will inherently be subjected to the same extraction process as WF. In some embodiments, the WSS 12 is operated to mix TW into WF before WF enters the feed side 31A or inside the MD unit 31. In other embodiments, the WSS 12 is operated to alternately supply TW and WF to the feed side 31A of the MD unit 31. Step 303C may be performed for other reasons than to increase the amount of EW, for example to mitigate membrane fouling. Membrane fouling involves accumulation of substances on the surface and/or within the pores of the membrane 31′ and results in deterioration of membrane performance. Step 303C may mitigate membrane fouling by diluting WF and/or rinsing the membrane 31′.

The utility of the water extraction technique described herein will be further explained in relation to a non-limiting numerical example for PD therapy. Generally, the total WF amount that is available for water extraction is correlated with the amount of extracted water, if all of the extracted water is provided to the dialysis system and ends up as WF. In the numerical example, it is assumed that 13.5 L (liters) of dialysis fluid is consumed during a PD session and that the dialysis fluid is generated by mixing a liquid concentrate and water at a volume ratio of 1:12.5, Thus, 12.5 L of water is consumed to produce the dialysis fluid. Assuming that 1 L of ultrafiltrate is extracted from the patient during the PD session, the resulting amount of spent dialysis fluid is 14.5 L. Further, it is assumed that 3.5 L of water is used as maintenance fluid (rinsing, disinfection, etc.), resulting in 3.5 L of spent maintenance fluid. Assuming that 75% of the water in the spent dialysis fluid and 90% of the spent maintenance fluid are extracted by operating the WSS 12 in the HDH mode, the amount of water that is extracted from the waste fluid (spent dialysis fluid and spent maintenance fluid) is 0.75*14.5+0.9*3.5≈14 L. The required amount of water is 12.5+3.5=16 L. Thus, in this numerical example, 14 L of the required 16 L of water is extracted from waste fluid. The remaining 2 L of water may be produced by net extraction of water from the surrounding air, by operating the WSS 12 in the DH mode and/or be provided to the WSS 12 as tap water. Thus, the main controller 60 may be configured to control the overall operation of the WSS 12 to achieve an EW target value of 16 L per day.

It is realized that the WSS 12 will significantly reduce, or even eliminate, the need to supply tap water before each therapy session performed by the dialysis system. It may be noted that a supply of fresh tap water may be made before a first therapy session in a sequence of therapy sessions, whereupon water is extracted from the waste fluid of the respective therapy session and used in this therapy session and/or in a subsequent therapy session. Alternatively, the WSS 12 may be operated in the DH mode to extract a required amount of water from surrounding air before the first therapy session. It is also realized that the amount of waste fluid to be discarded, corresponding to FWF in the drawings, is significantly reduced. In the numerical example above, 18 L of waste fluid is reduced to 0.25*14.5+0.1*3.5≈4 L. Clearly, the WSS 12 will facilitate disposal of the waste fluid that is generated by the dialysis system.

FIG. 4 is a block diagram of an example DHU sub-system 20 implemented as a unitary device. The DHU sub-system 20 comprises a water extraction unit 210, which is configured to receive and process the incoming gas stream (DIA) to change its phase state from gaseous to liquid, for at least part of the included moisture. Thereby, liquid water (EW) is extracted from the incoming gas stream (DIA), and the outgoing gas stream (DOA) with reduced humidity is generated.

In some embodiments, the water extraction unit 210 is configured to extract EW by direct condensation of the moisture in DIA, by cooling the gas below its dew point, optionally at elevated pressure. For example, the water extraction unit 210 may comprise a conventional cooling element, such as an evaporator coil, which is arranged to cool the DIA, causing water to condense. In these embodiments, box 210A represents the cooling element. This type of water extraction is mainly effective for DIA with high RH, such as above approximately 40%. Generally, the purity of EW obtained by this technique is dependent on the quality of the DIA.

In some embodiments, the water extraction unit 210 is instead configured to extract EW by use of a desiccant. In these embodiments, box 210A represents the desiccant. The desiccant is a hygroscopic substance which is arranged to interact with the DIA. During this interaction, the desiccant absorbs and/or adsorbs water molecules that are present in the DIA. The water extraction unit 210 is configured to process the desiccant 210A to release the water molecules, for example by one or more of heating, moisture vapor pressure change, or UV irradiation. The released water molecules are then collected to form EW. This type of water extraction is effective also for DIA with low RH, such as down to 20%, or even lower. Generally, the quality of EW obtained by this technique is dependent on the desiccant, in particular its selectivity towards water.

The DHU sub-system 20 in FIG. 4 comprises a gas inlet 202A, a gas outlet 202B, and a water outlet 202C. The gas inlet 202A opens to a gas inlet channel 201, which extends to the water extraction unit 210. A filter 202 is arranged in the gas inlet channel 201 for removal of particulate matter, such as debris, dust, etc., and possibly also volatile organic components, carbon, sub-micrometer particles, etc. A pumping device 203, for example a fan, is arranged downstream of the filter 202 to generate and drive a gas stream through the water extraction unit 210. A humidity sensor 204 is arranged in the gas inlet channel 201 to sense the inlet humidity Hdi of the DIA. The gas outlet 202B opens to a gas outlet channel 205, which extends from the water extraction unit 210. A humidity sensor 206 is arranged in the gas outlet channel 205 to sense the outlet humidity Hdo of the DOA. The water outlet 202C opens to a water outlet channel 207 which extends from the water extraction unit 210. A flow controller 208 is arranged in the water outlet channel 207 to control the flow of EW from the water extraction unit 210. The flow controller 208 may for example comprise a valve and/or a pumping device. A local controller (“control unit”) 209 is configured to generate control signals for the fan 203, the flow controller 208, and the water extraction unit 210, based on sensor signals from the humidity sensors 204, 206, to achieve one or more target values given by the control signal C3 from the main controller (60 in FIG. 2). For example, the control signal C3 may designate a target value for the flow rate of EW (“EW production rate”) and/or a target value for the outlet humidity Hdo. The target value for the EW production rate may be set to ensure that a sufficient amount of EW is extracted over a predefined time period, for example 24 hours. The local control unit 209 may, for example, control the flow rate of the DIA based on Hdi to achieve the target value of the EW production rate. Alternatively or additionally, the local control unit 209 may control the flow rate of the DIA based on Hdi to achieve the target value of Hdo.

The water extraction unit 210 may produce EW with sufficient purity for use in dialysis. Specifically, it has been found that an inherent purification of EW may be achieved by use of desiccant that has a high selectivity towards water. The high selectively implies that the desiccant is tailored to adsorb and/or absorb water molecules rather than other molecules that may be present in the DIA.

In some embodiments, the water extraction unit 210 is configured to produce EW that has conductivity of less than 10 μS/cm, and preferably less than 5 μS/cm or 1 μS/cm. As understood from the foregoing, this may be achieved by using a desiccant with a high selectivity of water. Under certain circumstances, a DHU sub-system 20 operating by direct condensation may also produce EW of sufficient purity.

In some embodiments, the desiccant is an ionic or covalent porous solid, including but not limited to metal-organic and organic porous framework materials, zeolites, organic ionic solids, inorganic ionic solids, organic molecular solids, or inorganic molecular solids, or any combination thereof. The desiccant may be used in a pure, single-phase form, as a composition of different active chemical materials, and/or in combination with performance enhancing additives modulating its properties. Performance enhancing additives may include materials with a high thermal conductivity and molar water absorptivity. The active chemical compound may be used in the form of a powders, extrudates, molded bodies, pressed pellets, pure or composite films, or sintered bodies. In some embodiments, the water capture material comprises an active chemical compound, such as a metal-organic framework (MOF). MOFs are porous materials that have repeating secondary building units (SBUs) connected to organic ligands. In some variations, the SBUs may include one or more metals or metal-containing complexes. In other variations, the organic ligands have acid and/or amine functional group(s). In certain variations, the organic ligands have carboxylic acid groups. Any MOF capable of adsorbing and desorbing water may be employed in the systems provided herein. In some embodiments, MOF-303 is used as desiccant. MOF-303 has a structure of Al(OH)(HPDC), where HPDC stands for 1H-pyrazole-3,5-dicarboxylate. Other conceivable MOFs for use as desiccant include, for example, MOF-801, MOF-841 and MIL-160. A combination of MOFs may also be used as desiccant. Further examples and implementation details are found in the articles “Metal-Organic Frameworks for Water Harvesting from Air”, by Kalmutzki et al., published in Adv. Mater. 2018, 30, 1704304, and “Practical water production from desert air”, by Fathieh et al., published in Sci. Adv. 2018, Vol. 8, Issue 6, which are incorporated herein by reference.

FIGS. 5A-5B illustrate embodiments of the WSS 12 in which the HU sub-system 30 comprises a recirculation path 34 that includes the feed side 31A of the MD unit 31. Thereby, the recirculation path 34 is arranged to allow for waste fluid to be recirculated on the feed side 31A of the MD unit 31 while gas is passed through the draw side 31B. By recirculating WF, the amount of water vapor that is transferred through the membrane 31′ may be increased for a given WF flow rate on the feed side 31A. The skilled person understands that to transfer a given amount of water from WF through the membrane 31′, the WF flow rate needs to be significantly lower when WF is passed only once on the feed side 31A, compared to when WF is circulated multiple times on the feed side 31A. A low WF flow rate may increase the risk of membrane fouling. Thus, the provision of a recirculation path 34 may improve the efficiency of the HU sub-system 30 and reduce the need for service and repair.

In the embodiments of FIGS. 5A-5B, a waste fluid pump is provided in the recirculation path 34 and operable to circulate WF on the recirculation path 34, and thus through the feed side 31A of the MD unit 31. The waste fluid pump corresponds to pump P1 in FIG. 2 and is represented by the same reference numeral. Further, like in FIG. 2, a WF sensor 33 is arranged downstream of the feed side 31A and configured to generate the signal ST. By analogy to the embodiment in FIG. 2, the operation of the pump 1 may be controlled based on the signal ST from the WF sensor 33.

The embodiments in FIGS. 5A-5B also include an improved heating arrangement (cf. 32 in FIG. 2), which includes a combination of an electrical heater 32′ and a heat transfer device 32″. The heat transfer device 32″ is arranged to transfer thermal energy from the humidified gas stream that is generated by the MD unit 31 to WF upstream of the feed side 31A of the MD unit 31. The heat transfer device 32″ may include a heat exchanger and/or a heat pump. Such a heat pump is operated to transfer thermal energy by use of a refrigeration cycle, as is well-known in the art. Alternatively or additionally, the heat transfer device 32″ may be configured to transfer thermal energy from the water extraction unit 210 in the DHU sub-system 20, given that thermal energy is released when water vapor is transformed into liquid water.

In the embodiments of FIGS. 5A-5B, the DHU sub-system 20 fluidly connected to receive humidified gas from the MD unit 31 on a first fluid path 23′ and to provide dehumidified gas to the MD unit 31 on a second fluid path 23″. A fluid path 26 for EW extends from the DHU sub-system 20 to an EW container 52, from which EW is provided to the dialysis system (not shown). An air outlet 24 is provided in the first fluid path 23′, and an air inlet 25 is provided in the second fluid path 23′. A respective on/off valve V1, V2 is arranged to selectively open and close the air outlet 24 and the air inlet 25. A further on/off valve V3 is arranged in the second fluid path 23″ downstream of the air outlet 24 to selectively open and close the second fluid path 23″. Functionally, the valves V1-V3 correspond to valves V′, V″ in FIG. 2 and define a valve arrangement for switching the WSS 12 between the HDH mode and the DH mode. In the HDH mode, valves V1, V2 are closed and valve V3 is open. In the DH mode, valves V1, V2 are open and valve V3 is closed. Assuming that the DHU sub-system 20 comprises a gas pumping device (cf. fan 203 in FIG. 4), air will be driven through the DHU sub-system 20 in both the HDH mode and the DH mode. In the DH mode, as shown by dashed arrows, EIA is admitted through the air inlet 25 and EOA is expelled through the air outlet 24.

The operation of the HU sub-system 30 in FIGS. 5A-5B is controlled by the main controller 60 (FIG. 2), optionally via a local controller 39 (“control unit”) as indicated in FIGS. 5A-5B. The local controller 39 may be configured to generate control signals for the pump P1, the heater 32′, heat transfer device 32″, as well as included valves and/or pumps as described below.

Turning now specifically to the embodiment in FIG. 5A, the HU sub-system 30 is configured for batch-wise processing of WF. The operating method of batch-wise processing is to introduce a batch of WF into the path 34, operate a waste fluid pump P1 to circulate the WF on the path 34, and thus through the feed side 31A, while gas is passed on the draw side 31B. The WF circulation is continued until the WF attains a predefined state. The predefined state is attained when the concentration-related property measured by the WF sensor 33 attains a limit value. This limit value determines the “water recovery ratio” of the HU sub-system 30, which denotes the proportion of the water in the WF that is available for transfer through the membrane 31. In the predefined state, the waste fluid forms “final waste fluid” (FWF), which is at least partly drained from the path 34. The amount of WF that is processed in each batch is given by the volume of the recirculation path 34. In the embodiment of FIG. 5A, a flow-through vessel 34A is included in the recirculation 34 to enable a large batch of WF to be processed. The HU sub-system 30 in FIG. 5A further comprises various flow controllers V4-V7, for example on/off valves, which are arranged to enable filling and draining of the recirculation path 34. Further, in the illustrated example, P1 is a bi-directional pump to be used for filling, circulation as well as draining. To fill the path 34, V4 is closed, V5 is opened and P1 is operated to pump WF into the path 34 towards the vessel 34A from an inlet line 35, which may extend to a WF container (51 in FIG. 2). When a sufficient quantity of WF has entered the path 34, V4 is opened, V5 is closed and P1 is reversed to circulate the WF in the path 34 through the vessel 34A and the MD unit 31. The amount of WF entering the path 34 may be monitored by any suitable means, for example a level sensor of the vessel 34A, a pressure sensor in the path 34, a flow meter, or by volumetric pumping by PT. To add tap water (TW), V7 is intermittently opened so that P1 draws TW from an inlet line 37, which may extend to a TW container (53 in FIG. 2). When the WF has attained the predefined state, V6 is opened and V4 is closed, so that P1 pumps the FWF into an outlet line 36, which may extend to an FWF container (not shown) or to a drain. In a variant, P1 is a one-directional pump, which is operable to circulate the WF on the path 34, and WF is pumped into the path by a separate pump arranged in the inlet line 35. The separate pump may be arranged downstream of the WF container (51 in FIG. 2). If no WF container is installed, the separate pump may be part of the dialysis system.

In the embodiment of FIG. 5B, the HU sub-system 30 is configured for continuous processing of WF. The operating method of continuous processing is to concurrently admit a first amount of WF into the path 34 and expel a second amount of processed WF from the path 34. The first and second amounts are jointly controlled so that the first amount replaces the second amount and the amount of water that is transported through the membrane 31′ from the feed side 31A to the draw side 31B. In other words, the difference between the first and second amounts is set substantially equal to the amount of water that leaves the path 34 in the MD unit 31. Various flow controllers are arranged to enable the filling and draining of the path 34. In the example of FIG. 5B, the flow controllers comprise a pump P2 in an inlet line 35, which may extend to a WF container (51 in FIG. 2), a pump P3 in an outlet line 36, which may extend to an FWF container (not shown) or to a drain, and an on/off valve V7 in an inlet line 37, which may extend to a TW container (53 in FIG. 2). In the HU sub-system 30 of FIG. 5B, P1 is operated to circulate WF in the path 34, while P2 is operated to supply WF and P3 is operated to remove WF, while maintaining an essentially constant amount of fluid in the path 34. The pumps P2, P3 may be jointly controlled in any suitable way, for example by maintaining a stable fluid pressure in the path 34 as measured by a pressure sensor, by volumetrically balancing the flow rates through paths 26, 35, 36. To add tap water, V7 is intermittently opened.

While the sub-systems for batch-processing and continuous processing differ by both structure and function, they are both operated based on the signal Si to cause a first flow controller to admit a first amount of WF into the recirculation path 34 and a second flow controller to expel a second amount of processed WF from the recirculation path 34, where the processed WF contains WF that has been recirculated through the feed side 31A of the MD unit 31 at least once. In FIG. 5A, the first flow controller corresponds to the combination of V4 and V5, and the second flow controller corresponds to the combination of V4 and V6. In FIG. 5B, the first flow controller corresponds to P2, and the second flow controller corresponds to P3.

In DH mode, the WSS 12 produces water by dehumidification of surrounding air. As explained earlier, the DH mode may be activated whenever WF is unavailable for water extraction, for example to close the gap between The target value and the amount of EW that is available for extraction from WF. The EW produced in DH mode is taken directly from the surrounding air. If a large amount of EW is produced in DH mode in a short time period, the humidity of the surrounding air may decrease to an unacceptable degree. On the other hand, if the amount of EW produced in DH mode is maximized, the amount of EW produced in HDH mode may be decreased. Thereby, in the HDH mode, the water recovery ratio may be lowered, which reduces the risk of membrane fouling. There is thus a trade-off between the risk of drying out the surrounding air, in the DH mode, and the risk of membrane fouling, in the HDH mode.

In some embodiments, the WSS 12 is operated to maximize the EW production rate in the DH mode while maintaining an acceptable humidity of the surrounding air. A first minimum limit may be defined for the humidity of the surrounding air, for example as measured by the sensor 204 in FIG. 2, and the DHU sub-system 20 may be operated to produce EW so as to not undercut the first minimum limit. In some embodiments, the first minimum limit is in the range of 20-40% RH. In some embodiments, a second minimum limit is defined for the air emitted to the surroundings (EOA), for example as measured by the sensor 206 in FIG. 2. In some embodiments, the second minimum limit is in the range of 0-20% RH. One or both of the first and second minimum limits may be used to control the WSS 12 in the DH mode. The respective minimum limit may be predefined for the WSS 12 or set by the user. The respective minimum limit may be scheduled to have different values at different times, for example to account for the anticipated presence of the user in the premises of the WSS 12. If required to exceed the minimum limit(s), the WSS 12 may be operated to reduce the air flow rate through the DHU sub-system 20, while maintaining a humidity difference between inlet humidity (Hdi in FIG. 2) and outlet humidity (Hdo in FIG. 2). In a variant, to exceed the minimum limit(s), the humidity difference is instead reduced and the air flow rate is maintained. In other variants, both air flow rate and humidity difference are adjusted.

In the HDH mode, all water vapor that enters the gas stream via the membrane 31′ may be harvested in the DHU sub-system 20. Thus, in some embodiments, the humidification rate in the HU sub-system 30 equals the dehumidification rate in the DHU sub-system 20, which in turn equals the EW production rate. In some situations, for example to avoid EW shortage, the WSS 12 may be operated in the HDH mode to increase EW production rate well above the average target. When doing so, one of the sub-systems 20, 30 will limit the EW production rate.

In some embodiments of the HDH mode, the DHU sub-system 20 acts as a master. This means that a required combination of flow rate and water content of the incoming gas stream (DIA) is determined to enable the DHU sub-system 20 to reach a target value of the EW production rate. The HU sub-system 30 is then operated to extract water from the waste fluid so as to achieve water content, for example given as inlet humidity (Hdi in FIG. 2) measured by sensor 204, while the fan 203 is operated to generate the required gas flow rate, optionally based on feedback from a flow meter (not shown). If it is unable to meet the required combination of water content and gas flow rate, the HU sub-system 30 may be operated to maximize its water transfer rate through the membrane 31′, which corresponds to maximizing the amount of water vapor that is provided to the DHU sub-system 20 per unit time. The water transfer rate may be adjusted by changing the inlet temperature and/or flow rate of the waste fluid on the feed side 31A, by changing the inlet temperature and/or flow rate of the gas on the draw side 31B, by changing the composition of the waste fluid (for example, spent dialysis fluid or maintenance fluid), by changing the water recovery ratio, or by any combination thereof.

The inlet temperature of the waste fluid on the feed side 31A may be attained by operating the heat transfer device 32″ to transfer heat to WF entering the feed side 31A from the gas leaving the draw side 31B. If necessary to attain a required inlet temperature, the electrical heater 32′ may be operated to increase the WF temperature, optionally based on feedback from a temperature sensor.

The WSS 12 may also be operated to reduce membrane fouling in the MD unit 31. This may be achieved by enhancing the near-surface shear forces by increasing the WF flow rate through the feed side 31A, by pre-diluting the WF with TW or a low-concentrated WF (if available) to lower the concentration of scaling compounds, or by alternatingly supply WF and TF (or high- and low-concentrated WF) through the feed side 31A. The risk of fouling generally increases with water recovery ratio. In some embodiments, the WSS 12 is operated at a water recovery ratio that results in an acceptable trade-off between fouling risk and water extraction.

Below follows a non-limiting use case for a combination of a WSS 12 and an APD system, which is operated to perform therapy sessions during nighttime. The starting point for the use case is the evening before a session. At the starting point, WF may or may not be available to the WSS 12. However, it is assumed that an EW container 52 in the WSS 12 holds, at the starting point, enough water to produce at least the first two fill volumes of PD fluid during the session. For example, the available amount of water may be 4-10 L. During PD fluid preparation, the initial fill phase and the initial dwell phase, the WSS 12 is operated to produce EW in DH mode if no WF is available, or in HDH mode if WF is available. After the initial drain phase and for the rest of the session, the WSS 12 is operated in HDH mode to produce EW from the spent PD fluid that is obtained in the respective drain phase. The produced EW is collected in the EW container 52, and the processed waste fluid (FWF) is discarded to a disposable container. Depending on the EW production rate in relation to the production rate of spent PD fluid during the session, spent PD fluid may be stored in the WF container 51 for subsequent processing. The WSS 12 may be occasionally switched to DH mode to utilize the water in the surrounding air without significantly impacting the humidity in the surroundings. After the last drain phase, the WSS 12 is operated to receive maintenance fluid from the APD system and to produce EW in the HDH mode from the spent PD fluid from the last drain phase, the maintenance fluid, and any saved WF. During daytime, the WSS 12 may be switched between the HDH mode and the DH mode, for example based on the humidity of the surrounding air. When user presence is detected during daytime, for example if the user interacts with the APD system, the user may be prompted to supply TW to the WSS 12 if deemed necessary based on the available amounts of EW and WF, the humidity of the surrounding air, the operating history of the APD system and/or the WSS 12, or any combination thereof.

It should be understood that the WSS 12 may be controlled based on either relative humidity or absolute humidity. For example, by also measuring temperature, a measured relative humidity may be converted into an absolute humidity.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1-27. (canceled)

28: A water supply system for a dialysis system, said water supply system comprising:
a first sub-system, which is configured to convert a first gas stream into a second gas stream by extracting liquid water from the first gas stream, and provide the liquid water for use by the dialysis system,
a second sub-system, which is configured to process the second gas stream, by use of waste fluid from the dialysis system, to generate the first gas stream with increased humidity compared to the second gas stream, and
a control arrangement, which is configured to jointly operate the first and second sub-systems to generate a target amount of said liquid water,
wherein the second sub-system comprises a membrane distillation, MD, unit that defines a feed side and a draw side separated by a hydrophobic membrane, wherein the MD unit is arranged to receive the waste fluid at an inlet on the feed side, and receive the second gas stream at an inlet on the draw side, and wherein the MD unit is configured to generate the first gas stream by transporting water vapor from the waste fluid into the second gas stream through the hydrophobic membrane via a difference in partial water vapor pressure between the feed side and the draw side.

29: The water supply system of claim 28, wherein the control arrangement is configured to selectively operate the first sub-system to obtain at least part of the first gas stream from surrounding air, and supply at least part of the second gas stream to the surrounding air.

30: The water supply system of claim 28, wherein the control arrangement is configured to selectively switch the system between a first mode, in which the system is configured to transfer the first and second gas streams between the first and second sub-systems in a closed loop, and a second mode, in which the system is configured to block said transfer and operate the first sub-system to obtain the first gas stream from the surrounding air and provide the second gas stream to the surrounding air.

31: The water supply system of claim 30, wherein the control arrangement is configured to switch between the first and second modes based on at least one of a current water content of the surrounding air, availability of the waste fluid, availability of said liquid water, or a time schedule.

32: The water supply system of claim 28, wherein the second sub-system further comprises a heating arrangement which is arranged upstream of the inlet on the feed side of the MD unit and is operable to heat the waste fluid.

33: The water supply system of claim 32, wherein the heating arrangement comprises a heat transfer device, which is arranged to transfer thermal energy from the first gas stream, as generated by the MD unit, to the waste fluid.

34: The water supply system of claim 28, wherein the second sub-system further comprises a WF sensor, which is arranged downstream of an outlet on the feed side of the MD unit to provide a measurement signal indicative of a concentration-related property of the waste fluid, and wherein the control arrangement is configured to operate the second sub-system based on the measurement signal.

35: The water supply system of claim 34, wherein the concentration-related property comprises a concentration, a density, a conductivity, a color, a transparency, or a refractive index.

36: The water supply system of claim 34, wherein the second sub-system defines a recirculation path that includes the feed side of the MD unit, wherein the second sub-system comprises a pumping device in the recirculation path, and wherein the control arrangement is configured to operate the pumping device, based on the measurement signal, to recirculate the waste fluid through the feed side of the MD unit.

37: The water supply system of claim 36, wherein the control arrangement is further configured to, based on the measurement signal, selectively operate a first flow controller to admit a first amount of waste fluid into the recirculation path and a second flow controller to expel a second amount of processed waste fluid from the recirculation path, wherein the processed waste fluid contains waste fluid that has been recirculated at least once through the feed side of the MD unit.

38: The water supply system of claim 37, wherein the control arrangement is configured to, in sequence, operate the first flow controller to admit the first amount into the recirculation path, operate the pumping device to circulate at least the first amount through the feed side of the MD unit, and operate the second flow controller to expel the second amount from the recirculation path.

39: The water supply system of claim 37, wherein the control arrangement is configured to, concurrently, operate the first flow controller to admit the first amount into the recirculation path and the second flow controller to expel the second amount from the recirculation path so that a difference between the first and second amounts substantially equals a third amount of water transported into the second gas stream through the hydrophobic membrane.

40: The water supply system of claim 36, wherein the control arrangement is configured to selectively operate a supply arrangement to supply tap water to the recirculation path and operate the pumping device to circulate the tap water through the feed side of the MD unit.

41: The water supply system of claim 28, further comprising an EW container, which is arranged to receive the liquid water from the first sub-system, wherein the control arrangement is configured to operate the first and second sub-systems in dependence of a fill level of the EW container, as indicated by a level sensor associated with the EW container.

42: The water supply system of claim 28, further comprising a WF container, which is arranged for intermediate storage of the waste fluid and is fluidly connected to the inlet on the feed side of the MD unit.

43: The water supply system of claim 42, wherein the WF container is associated with a sterilization device which is operable to sterilize the WF container.

44: The water supply system of claim 28, wherein the first and second gas streams comprise air.

45: The water supply system of claim 28, wherein the control arrangement is configured to determine first settings of the first sub-system to achieve the target amount, and determine second settings of the second sub-system based on the first settings, wherein the second settings define a water content and a flow rate of the first gas stream as generated by the second sub-system.

46: An arrangement comprising the water supply system of claim 28, and the dialysis system which is configured to receive medical fluid for use in dialysis therapy performed by the dialysis system and to produce the waste fluid that is at least partly generated from the medical fluid during the dialysis therapy, wherein the dialysis system is fluidly connected to transfer the waste fluid to the water supply system.

47: A computer-implemented method of providing water for use by a dialysis system, said method comprising:
operating a first sub-system to convert a first gas stream into a second gas stream by extracting liquid water from the first gas stream;
providing the liquid water for use by the dialysis system; and
operating a second sub-system, in coordination with the first sub-system, to process the second gas stream, by use of waste fluid from the dialysis system, to generate the first gas stream with increased humidity compared to the second gas stream;
wherein said operating the second sub-system comprises: supplying the waste fluid at an inlet on a feed side of a membrane distillation, MD, unit, and supplying the second gas stream at an inlet on a draw side of the MD unit, the draw side being separated from the feed side by a hydrophobic membrane, the MD unit being configured to generate the first gas stream by transporting water vapor from the waste fluid into the second gas stream through the hydrophobic membrane via a difference in partial water vapor pressure between the feed side and the draw side.
